(19) United States
(12) Patent Application Publication
(10) Pub. No.: US 2025/0041329 A1
(43) Pub. Date: Feb. 6, 2025

(54) ACIDIC POLYMER COMPOSITIONS AND METHODS OF USE THEREOF

(71) Applicant: NexEos Diagnostics, Inc., Pottstown, PA (US)

(72) Inventors: Gerald J. GLEICH, Salt Lake City, UT (US); Evan CUTLER, Salt Lake City, UT (US)

(73) Assignee: NexEos Diagnostics, Inc., Pottstown, PA (US)

(21) Appl. No.: 18/794,466

(22) Filed: Aug. 5, 2024

Related U.S. Application Data
(60) Provisional application No. 63/517,723, filed on Aug. 4, 2023.

(51) Int. Cl. A61K 31/785; A61K 47/59; A61K 51/06; A61P 29/00

(52) U.S. Cl. CPC A61K 31/785; A61K 47/595; A61K 51/065; A61P 29/00

(57) ABSTRACT

A method of treating a tissue exhibiting eosinophil-related inflammation in a subject is disclosed. The method includes administering a composition to the subject that contains a therapeutically effective amount of a polymer and a pharmaceutically acceptable excipient. The polymer is formed from an acidic amino acid, e.g., aspartate and/or glutamate, or a pharmaceutically acceptable salt thereof. The polymer has an average molecular weight of at least about 5 kDa. The polymer binds to one or more eosinophil granule proteins in the tissue to neutralize the eosinophil granule proteins and treat the eosinophil-related inflammation.

### CROSS-REFERENCE TO RELATED APPLICATION

This application claims the benefit of priority to U.S. Provisional Application No. 63/517,723 entitled “Acidic Polymer Compositions and Methods of Use Thereof,” filed Aug. 4, 2023, which is incorporated herein by reference in its entirety.

### GOVERNMENT RIGHTS

This invention was made with government support under Grant No. 5R43AI162331-02 awarded by the National Institutes of Health (NIH). The government has certain rights in the invention.

### SUMMARY

The present disclosure relates generally to compositions and methods of inhibiting the activity of eosinophil granule proteins on a tissue. More particularly, the present disclosure relates to compositions and methods for binding to eosinophil granule proteins at a site of eosinophil-related inflammation. The methods may be applied to, for example, imaging a site of eosinophil-related inflammation, diagnosing eosinophil-related inflammation, and/or treating eosinophil-related inflammation in a subject.

Embodiments of the present subject matter are directed to methods of treating a tissue exhibiting eosinophil-related inflammation in a subject, comprising: administering to the subject a composition comprising a therapeutically effective amount of a polymer of an acidic amino acid and a pharmaceutically acceptable excipient, the polymer having an average molecular weight of at least about 5 kDa, wherein the polymer binds to one or more eosinophil granule proteins in the tissue to treat the eosinophil-related inflammation.

Embodiments of the present subject matter are also directed to methods of reducing eosinophil-related inflammation in a tissue in a subject, comprising: administering to the subject a composition comprising a therapeutically effective amount of a polymer of an acidic amino acid and a pharmaceutically acceptable excipient, the polymer having an average molecular weight of at least about 5 kDa, wherein the polymer binds to one or more eosinophil granule proteins in the tissue to reduce the eosinophil-related inflammation.

Embodiments of the present subject matter are also directed to compositions for use in treating a tissue exhibiting eosinophil-related inflammation in a subject, comprising: an effective amount of a polymer of an acidic amino acid, the polymer having an average molecular weight of at least about 5 kDa, wherein the polymer is configured to bind to one or more eosinophil granule proteins in the tissue to treat the eosinophil-related inflammation; and a pharmaceutically acceptable excipient.

Embodiments of the present subject matter are also directed to methods of producing a medical image of an organ in a subject, comprising: administering, to the subject, a composition comprising an effective dose of a radiolabeled polymer of an acidic amino acid and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and detecting the radiolabeled polymer/eosinophil granule protein complex in a mucosal tissue of the organ, thereby producing a medical image of the organ.

Embodiments of the present subject matter are also directed to methods of diagnosing eosinophil-related inflammation in an organ of a subject, comprising: administering, to the subject, a composition comprising an effective dose of a radiolabeled polymer of an acidic amino acid and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and detecting the radiolabeled polymer/eosinophil granule protein complex in a mucosal tissue of the organ, thereby diagnosing the eosinophil-related inflammation in the organ.

In some embodiments, the radiolabeled polymer/eosinophil granule protein complex is detected using single-photon emission computed tomography (SPECT), positron emission tomography (PET), conventional or computed tomography (CT), magnetic resonance imaging (MRI), or a combination thereof.

In some embodiments, the acidic amino acid is selected from the group consisting of aspartate, glutamate, and combinations thereof. In additional embodiments, the polymer of glutamate is selected from the group consisting of a poly-L-glutamic acid, a poly-D-glutamic acid, Y polyglutamic acid, and combinations thereof. In additional embodiments, the polymer of aspartate is α poly-L-aspartic acid.

In some embodiments, the one or more eosinophil granule proteins comprise one or more of major basic protein 1 (eMBP1), major basic protein 2 (eMBP2), eosinophil derived neurotoxin (EDN), eosinophil cationic protein (ECP), and eosinophil peroxidase (EPO).

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. Various aspects of at least one example are discussed below with reference to the accompanying drawings, which are not intended to be drawn to scale. In the drawings:

FIG. 1 depicts the potencies of several forms of aspartate for inhibiting eMBP1 in accordance with an embodiment.

FIG. 2 depicts the half-maximal inhibitory concentration (IC50) for inhibiting eMBP1 as a function of molecular weight for each of several acidic polymers in accordance with an embodiment.

FIG. 3A depicts inhibition of eMBP1-stimulated histamine release to different degrees by glycosylated pro-piece from HEK293 cells and unfractionated heparin as compared to a control protein, human serum albumin (HSA) in accordance with an embodiment.

FIG. 3B depicts inhibition of eMBP1-stimulated histamine release to different degrees by glycosylated pro-piece from HEK293 cells, unglycosylated pro-piece from E. Coli, unfractionated heparin, and polyglutamic acid in accordance with an embodiment.

### DETAILED DESCRIPTION

This disclosure is not limited to the particular systems, devices and methods described, as these may vary. Moreover, the processes, compositions, and methodologies described in particular embodiments are interchangeable. Therefore, for example, a composition, dosage regimen, route of administration, and so on described in a particular embodiment may be used in any of the methods described in other particular embodiments. The terminology used in the description is for the purpose of describing the particular versions or embodiments only, and is not intended to limit the scope. Such aspects of the disclosure may be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth. In a further example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg and ranges within, such as 2 mg to 5 mg, 3 mg to 5 mg, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

All percentages, parts and ratios of a composition are based upon the total weight of the composition and all measurements made are at about 25° C., unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “improves” is used to convey that the present invention changes either the form, characteristics, structure, function, and/or physical attributes of the tissue to which it is being provided, applied, or administered. “Improves” may also refer to the overall physical state of an individual to whom an active agent has been administered. For example, the overall physical state of an individual may “improve” if one or more symptoms of the disease, condition or disorder are alleviated by administration of an active agent.

The terms “patient” and “subject” are interchangeable and refer to any living organism which contains neural tissue. As such, the terms “patient” and “subject” may include, but are not limited to, any non-human mammal, primate, or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child, or infant.

In some embodiments herein, the methods may be utilized with or on a subject in need of such treatment, which may also be referred to as “in need thereof.” The phrase “in need thereof” means that the subject has been identified as having a need for the particular method or treatment and that the treatment is being given to the subject for that particular purpose.

Where this disclosure makes reference to the term “doctor” and additional terms for various medical professionals by specific job title or role, nothing in this disclosure is intended to be limited to a specific job title or function. Doctors or medical professionals can include any doctor, nurse, medical professional, or technician. Any of these terms or job titles can be used interchangeably with the user of the systems disclosed herein unless otherwise explicitly demarcated. For example, a reference to a physician could also apply, in some embodiments to a technician, nurse, or other health care provider.

The term “tissue” refers to any aggregation of similarly specialized cells which are united in the performance of a particular function.

The term “diagnose,” “diagnosing,” or “diagnosis” as used herein refers to the process of identifying the existence and/or nature of a disease, condition, or other physiological state in a subject from its characteristics, signs, and symptoms. Diagnosis may include a statement or conclusion related to the disease, disorder, condition, or other physiological state in the subject based on such a process.

The term “treat,” “treated,” or “treating” as used herein refers to both therapeutic treatment, wherein the object is to reduce the frequency of, or delay the onset of, symptoms of a medical condition or physiological state, or to otherwise obtain beneficial or desired clinical results. For the purposes of this invention, beneficial or desired clinical results include, but are not limited to, reversal, reduction, or alleviation of symptoms of a condition or physiological state; diminishment of the extent of the condition, physiological state, disorder or disease; stabilization (i.e., not worsening) of the state of the condition, physiological state, disorder or disease; delay in onset or slowing of the progression of the condition, physiological state, disorder or disease; amelioration of the condition, physiological state, disorder or disease state; and remission (whether partial or total), whether detectable or undetectable, or enhancement or improvement of the condition, physiological state, disorder or disease. Treatment includes eliciting a clinically significant response without excessive levels of side effects. In some embodiments, treatment or reduction of eosinophil-related inflammation as referred to in the present disclosure may comprise treating or reducing swelling (i.e., tumour), redness or erythema (i.e., rubor), heat or warmth (i.e., calor), pain (i.e., dolor), and/or dysfunction (i.e., function laesa) associated with the inflammation and/or any additional effects of the eosinophil granule proteins on the tissue. For example, where the eosinophil-related inflammation is skin inflammation (e.g., dermal), treating or reducing the inflammation may comprise treating or reducing swelling, redness, heat, and/or pain associated with the skin inflammation. In another example, where the eosinophil-related inflammation is nasal inflammation, treating or reducing the inflammation may comprise treating or reducing rhinorrhea, sneezing, obstruction, and/or pain associated with the nasal inflammation. In another example, where the eosinophil-related inflammation is ocular inflammation, treating or reducing the inflammation may comprise treating or reducing erythema, swelling, pruritus, and difficulty seeing (i.e., loss of function) associated with the ocular inflammation.

The term “disorder” is used in this disclosure to mean, and is used interchangeably with, the terms disease, condition, or illness, unless otherwise indicated.

Any reference to symptoms, signs, or biomarkers are herein collectively referred to as “symptom.”

As used herein, the term “therapeutic” means an agent utilized to treat, combat, ameliorate, or improve an unwanted condition, physiological state, or disease of a patient. In part, embodiments of the present invention are directed to the improving an unwanted condition such as an untimely labor during pregnancy.

The term “effective amount” is employed herein to refer to an amount of a compound that, when administered to a subject, is appropriate for carrying out a purpose of the compound. The actual amount which comprises the “effective amount” will vary depending on a number of conditions including, but not limited to, the severity of the condition or physiological state, the size and health of the patient, and the route of administration. A skilled medical practitioner can readily determine the appropriate amount using methods known in the medical arts.

The term “therapeutically effective amount” is employed herein to refer to an amount of a compound that, when administered to a subject, is capable of reducing a symptom of a condition, physiological state, or disorder in a subject. The actual amount which comprises the “therapeutically effective amount” will vary depending on a number of conditions including, but not limited to, the severity of the condition or physiological state, the size and health of the patient, and the route of administration. A skilled medical practitioner can readily determine the appropriate amount using methods known in the medical arts.

The phrase “pharmaceutically acceptable” is employed herein to refer to those agents of interest/compounds, salts, compositions, dosage forms, etc., which are within the scope of sound medical judgment and suitable for use in contact with the tissues of human beings and/or other mammals without excessive toxicity, irritation, allergic response, or other problem or complication, commensurate with a reasonable benefit/risk ratio. In some embodiments, pharmaceutically acceptable means approved by a regulatory agency of the federal or a state government, or listed in the U.S. Pharmacopeia or other generally recognized pharmacopeia for use in mammals (e.g., animals), and more particularly, in humans.

The terms “administer,” “administering” or “administration” as used herein refer to administering to a subject a compound (also referred to as an agent of interest), a pharmaceutically acceptable salt of the compound (agent of interest), or a composition directly by the subject or by a health care provider. “Administering” when used in conjunction with a therapeutic means to administer a therapeutic directly or indirectly into or onto a target tissue to administer a therapeutic to a patient whereby the therapeutic positively impacts the tissue to which it is targeted. “Administering” may include the act of self-administration or administration by another person such as a health care provider.

The term “inhibiting” includes the administration of a composition of the present invention to prevent the onset of a physiological state (e.g., labor in pregnancy); alleviating or reducing the symptoms and/or characteristics associated with the physiological state; delaying, decreasing, or interrupting the progression of the physiological state and/or its symptoms and/or characteristics; or eliminating the physiological state.

The compositions produced by the methods of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration (either alone or in combination with other pharmaceuticals) can be, but are not limited to, sublingual, injectable (including short-acting, depot, implant and pellet forms injected subcutaneously or intramuscularly), topical (including nasal sprays, ointments, or creams, e.g., for application to the skin), and/or by use of vaginal creams, suppositories, pessaries, vaginal rings, rectal suppositories, intrauterine devices, and transdermal forms such as patches and creams.

Specific modes of administration will depend on the indication or purpose. The selection of the specific route of administration and the dose regimen is to be adjusted or titrated by the clinician according to methods known to the clinician in order to obtain the optimal clinical response. The amount of compound to be administered is that amount which is effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

For oral administration, compositions can be formulated readily by combining agents of interest with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient to be imaged, diagnoses, and/or treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Pharmaceutical compositions which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Capsules may also be coated with additional layers to protect the contents through one or more phases of digestion and/or delay release of the contents. For example, the capsules or other carriers may include an enteric coating (e.g., formed by a polymer) to prevent dissolution or disintegration in the gastric environment. All compositions for oral administration should be in dosages suitable for such administration.

The term “carrier” as used herein encompasses carriers, excipients, and diluents, meaning a material, composition, or vehicle, such as a liquid or solid filler, diluent, excipient, solvent, or encapsulating material involved in carrying or transporting a pharmaceutical, cosmetic or other agent across a tissue layer such as the stratum corneum or stratum spinosum. Pharmaceutical compositions of the compounds may also comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

As used herein, a “mucosal tissue” is a tissue lining various cavities within the body. Examples of a mucosal tissue include, but are not limited to, mucosal tissue lining the nose, sinuses, bronchi, lungs, conjunctiva, oral cavity, tongue, esophagus, stomach, pylorus, duodenum, jejunum, ileum, ascending colon, caecum, appendix, transverse colon, descending colon, rectum, anus, urethra, and urinary bladder. A mucosal tissue comprises an epithelial surface, glandular epithelium which secretes mucus, basement membrane, and submucosa with connective tissue.

As used herein, an “eosinophil granule protein” is a protein that comprises the granules in eosinophils. When an eosinophil is activated, granule proteins are released from the cell into the surrounding tissue. The released granule proteins can cause pathologic inflammatory responses in the surrounding tissue, for example esophageal mucosal tissue. Examples of eosinophil granule proteins include, but are not limited to, major basic protein (MBP), major basic protein 1 (MBP-1), major basic protein 2 (MBP-2), eosinophil derived neurotoxin (EDN), eosinophil cationic protein (ECP), and eosinophil peroxidase (EPO). Other examples of eosinophil granule proteins are provided in Kita et al., Biology of Eosinophils, Chapter 19 of Immunology, which is hereby incorporated by reference for its teaching of examples of eosinophil granule proteins.

As used herein, “high molecular weight heparin” refers to heparin and/or heparin salts (e.g., heparin sodium) having a molecular weight of about 20 kDa or greater. Heparin polymer typically consists of a mixture of polydisperse linear polymers, i.e., having molecular chains of varying lengths, such that the molecular weight of the heparin chains varies and cannot be fully described by a single number. Accordingly, high molecular weight heparin is more particularly described as having an average molecular weight of about 20 kDa or greater. Average molecular weight may be calculated as a weight average (i.e., the summation of all represented molecular weights multiplied by their weight fraction in the sample) and/or a number average (i.e., total weight of the sample divided by the number of molecules in the sample). Furthermore, high molecular weight heparin may have a different polydispersity than unfractionated heparin as further described herein. Polydispersity may be quantified as a polydispersity index (PDI):

$$PDI = \frac{{M}_{W}}{{M}_{N}}$$

where M_{W} is the weight average molecular weight of the sample (i.e., the sum of each molecule's molecular weight multiplied by the molecule's weight fraction of the total sample's weight) and M_{N} is the number average molecular weight of the compound. In some instances, high molecular weight heparin may have a lower polydispersity than unfractionated heparin. In some instances, high molecular weight heparin may have a higher polydispersity than unfractionated heparin. In other instances, high molecular weight heparin may have a substantially similar polydispersity to unfractionated heparin.

As used herein, “low molecular weight heparin” refers to heparin and/or heparin salts (e.g., heparin sodium) having a molecular weight of about 8 kDa or less. For example, Enoxaparin is a product in a low molecular weight heparin family and has a molecular weight of about 4.5 kDa. Heparin polymer typically consists of a mixture of polydisperse linear polymers, i.e., having molecular chains of varying lengths, such that the molecular weight of heparin chains varies and cannot be fully described by a single number. Accordingly, low molecular weight heparin is more particularly described as having an average molecular weight of less than about 8 kDa. Average molecular weight may be calculated as a weight average (i.e., the summation of all represented molecular weights multiplied by their weight fraction in the sample) and/or a number average (i.e., total weight of the sample divided by the number of molecules in the sample). Furthermore, the polydispersity of low molecular weight heparin may vary based on the method of depolymerization. In some instances, the low molecular weight heparin may have a lower polydispersity than unfractionated heparin as further described herein. In other cases, low molecular weight heparin may have a polydispersity substantially equal to and/or greater than unfractionated heparin.

As used herein, “unfractionated heparin” or “heparin” refers to a heparin polymer with molecular chains of varying lengths, and molecular weights ranging from 3 to 30 kDa. “Unfractionated heparin” or “heparin” may have a greater polydispersity than high molecular weight heparin or low molecular weight heparin, not having been fractionated to sequester the fraction of molecules with a particular limited range of molecular weight. In other instances, unfractionated heparin may have a lower or substantially equal polydispersity to high molecular weight heparin or low molecular weight heparin.

Additional polymers and compositions may also be described by “average molecular weight” throughout the present disclosure. Polymer compositions can typically contain molecular chains of varying lengths, such that the molecular weight of the polymers in the composition have at least some variation and cannot be fully described by a single number. Accordingly, the polymers may be more particularly described by an average molecular weight (e.g., 5 kDa or greater as discussed throughout the present disclosure). As used herein, the term “average molecular weight” may be calculated as a weight average (i.e., the summation of all represented molecular weights multiplied by their weight fraction in the sample) and/or a number average (i.e., total weight of the sample divided by the number of molecules in the sample).

As used herein, a “radiolabel” is an isotopic composition that can be attached to a substance, for example a polymer, to track the substance as it passes through a system or tissue. A non-limiting example of a radiolabeled substance is a radiolabeled acidic polymer including, but not limited to radiolabeled polyaspartic acid, radiolabeled polyglutamic acid, and radiolabeled carboxymethylcellulose. In some embodiments, a radiolabeled polymer can be ^{99m}Tc-PASA and/or ^{99m}Tc-PGA. Examples of other radiolabels include, but are not limited to, 111 In, 14C, 3H, 13N, 18F, 51Cr, 125I, 133Xe, 81mKr, and 131I. Other radiolabels that can be attached to a substance, for example an acidic polymer, can be found in Table 1. A radiolabel, for example, ^{99m}Tc, can be attached to a substance, for example an acidic polymer, using commercially available reagents well known to persons of ordinary skill in the art.

TABLE 1
Commonly utilized radiolabels.

| Nuclide | Physical half-life | |
|---|---|---|
| ³H | 12.3 | years |
| ¹¹C | 20.4 | minutes |
| ¹³N | 10 | minutes |
| ¹⁴C | 5730 | years |
| ¹⁵O | 2 | minutes |
| ¹⁸F | 110 | minutes |
| ³²P | 14.3 | days |
| ⁵¹Cr | 27.7 | days |
| ⁵²Fe | 8.3 | hours |
| ⁵⁷Co | 271 | days |
| ⁵⁸Co | 71 | days |
| ⁵⁹Fe | 45 | days |
| ⁶⁰Co | 5.2 | years |
| ⁶²Zn | 9.3 | hours |
| ⁶²Cu | 9.7 | minutes |
| ⁶⁴Cu | 12.7 | hours |
| ⁶⁷Cu | 2.6 | days |
| ⁶⁷Ga | 78.2 | hours |
| ⁶⁸Ga | 68 | minutes |
| ⁷⁶Br | 16 | hours |
| ^{81m}Kr | — | |
| ⁸²Rb | 75 | seconds |
| ⁸²Sr | 25.5 | days |
| ⁸⁶Y | 14.74 | hours |
| ⁸⁹Zr | 3.27 | days |
| ⁸⁹Sr | 50.6 | days |
| ⁹⁰Sr | 28.5 | years |
| ⁹⁰Y | 2.7 | days |
| ^{99m}o | 66 | hours |
| ^{99m}Tc | 6.0 | hours |
| ¹¹¹In | 2.8 | days |
| ¹¹³In | 100 | minutes |
| ¹²³I | 13.2 | hours |
| ¹²⁴I | 4.2 | days |
| ¹²⁵I | 60 | days |
| ¹³¹I | 8.0 | days |
| ¹³³Xe | 5.3 | days |
| ¹³⁷Cs | 30 | years |
| ¹⁵³Sm | 1.9 | days |
| ¹⁸⁶Re | 3.8 | days |
| ²⁰¹Tl | 73 | hours |

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

### Eosinophils and Eosinophil Granule Proteins

The eosinophil is a peripheral blood leukocyte containing an abundance of cytoplasmic granules, rich in cationic protein toxins. Among these, the most abundant on a molar basis is the eosinophil major basic protein-1 (eMBP1), which comprises the crystalloid core of the eosinophil granule and is a 13.8 kDa single polypeptide. Eosinophil granule proteins, including eMBP1, are toxic and highly basic.

While studies have elucidated the nature of eMBP1 and some of its functions, the entirety of eMBP1's biological purpose is still not fully understood. eMBP1 is released by activated eosinophils, eMBP1 drives inflammation and clinical symptomatology in subjects. It also remains in target tissue much longer than eosinophils and it can be considered a “footprint” of eosinophils. It is understood that eMBP1 kills helminths, bacteria, and numerous cells, such as respiratory epithelium, but also activates cells, including basophils and mast cells. Studies of human diseases show that eMBP1 is present in secretions from patients with eosinophil-mediated diseases, including asthma, chronic rhinosinusitis, chronic sinus inflammation, allergic inflammation, and gastrointestinal diseases, and it is deposited on damaged targets. These studies show that the eosinophil mediates its damage to parasites and tissues by discharging its toxin rich granules onto microbial targets and tissues.

Physiologically, eMBP1 is synthesized in a precursor form known as pro-eosinophil major basic protein 1 (pro-eMBP1), which is composed of eMBP1 and a remarkably acidic pro-piece sequence. Developing eosinophils synthesize pro-eMBP1, and the pro-piece is removed during granule maturation in the eosinophilic leukocyte to convert pro-eMBP1 to eMBP1. The amino acid sequences of pro-eMBP1, eMBP1, and the pro-piece are disclosed in further detail in International Patent Application No. PCT/US2022/075946 entitled “Compositions and Methods for Diagnosing, Detecting, and Treating Eosinophil-Related Diseases,” filed on Sep. 2, 2022, which is hereby incorporated herein by reference in its entirety.

eMBP1 is stored in the eosinophil granule as a distinctive part of the granule, referred to as the granule core. When stored in the granule core, eMBP1 is inactive, but once released, it is a potent cytotoxin and cytostimulant capable of activating cells.

### High Molecular Weight Acidic Polymers for Binding Eosinophil Granule Proteins

As discussed herein, highly acidic substances such as aspartic acid (also referred to herein as aspartate), glutamic acid (also referred to herein as glutamate), and/or polymer forms thereof may be capable of binding eosinophil granule proteins (EGPs). Conventional acidic substances for use in binding to EGPs are disclosed in further detail in U.S. Pat. No. 5,250,293 entitled “Method for the Treatment of Hypersensitivity Diseases by Administration of Anionic Polymers,” filed on Apr. 22, 1991, and issued on Oct. 5, 1993, which is hereby incorporated herein by reference in its entirety. Despite this capability, these acidic substances have not emerged as favored agents for localizing to sites of eosinophil-related inflammation because it is not evident that they are capable of localizing to eosinophil-related inflammation with substantial specificity and avidity, i.e., substantially similar to or greater than other known EGP inhibitors such as heparin. Furthermore, the precise form and structure of the acidic substances may alter the specificity and/or avidity for eosinophil granule proteins, thereby affecting the dosing and/or the overall practicality of the acidic substances for this purpose.

In embodiments herein, it is submitted that high molecular weight polyamino acids (e.g., polyaspartic acid, polyglutamic acid, and other polyacids) may be particularly effective for localizing to sites of eosinophil-related inflammation. Furthermore, the high molecular weight (HMW) forms of polyaspartic acid (PASA) and polyglutamic acid (PGA) may also be effective for neutralizing the toxic effects of eMBP1 and other EGPs including eMBP2, EDN, ECP, and EPO. In some embodiments, the HMW acidic polymers can function as a medication by application to or delivery to one or more sites of eosinophil-related inflammation. Furthermore, because the HMW acidic polymers can be used to target eosinophil-related inflammation, tracers and/or therapeutic agents may be conjugated to the HMW acidic polymers to provide a targeted delivery to the eosinophil-related inflammation. HMW acidic polymers may be advantageous because they may bind eosinophil granule proteins more avidly than other known EGP inhibitors. Accordingly, a lower quantity or dose of the acidic polymer may be required as compared to other EGP inhibitors in order to achieve effective localization to eosinophil-related inflammation and/or therapeutic effect thereon because a greater percentage of the administered dose will localize to the sites of inflammation. In some cases, HMW acidic polymers may also be easier to produce than other known EGP inhibitors.

Embodiments herein are directed to methods of treating a tissue exhibiting eosinophil-related inflammation in a subject. The method comprises administering to the subject a composition comprising a therapeutically effective amount of a polymer of an acidic amino acid or a pharmaceutically acceptable salt thereof and a pharmaceutically acceptable excipient, the polymer having an average molecular weight of at least about 5 kDa, wherein the polymer binds to one or more eosinophil granule proteins in the tissue to treat the eosinophil-related inflammation. In some embodiments, the acidic amino acid is selected from the group consisting of aspartate, glutamate, and combinations thereof.

Embodiments herein are also directed to methods of reducing eosinophil-related inflammation in a tissue in a subject. The method comprises administering to the subject a composition comprising a therapeutically effective amount of a polymer of an acidic amino acid or a pharmaceutically acceptable salt thereof and a pharmaceutically acceptable excipient, the polymer having an average molecular weight of at least about 5 kDa, wherein the polymer binds to one or more eosinophil granule proteins in the tissue to reduce the eosinophil-related inflammation. In some embodiments, the acidic amino acid is selected from the group consisting of aspartate, glutamate, and combinations thereof.

Embodiments herein are also directed to a composition for use in treating a tissue exhibiting eosinophil-related inflammation in a subject. The composition comprises an effective amount of a polymer of an acidic amino acid or a pharmaceutically acceptable salt thereof, and a pharmaceutically acceptable excipient. The polymer has an average molecular weight of at least about 5 kDa, wherein the polymer is configured to bind to one or more eosinophil granule proteins in the tissue to treat the eosinophil-related inflammation. In some embodiments, the acidic amino acid is selected from the group consisting of aspartate, glutamate, and combinations thereof.

In some embodiments, treatment or reduction of eosinophil-related inflammation as referred to in the present disclosure may comprise treating or reducing swelling (i.e., tumour), redness or erythema (i.e., rubor), heat or warmth (i.e., calor), pain (i.e., dolor), and/or dysfunction (i.e., function laesa) associated with the inflammation and/or any additional effects of the eosinophil granule proteins on the tissue. For example, where the eosinophil-related inflammation is skin inflammation (e.g., dermal), treating or reducing the inflammation may comprise treating or reducing swelling, redness, heat, and/or pain associated with the skin inflammation. In another example, where the eosinophil-related inflammation is nasal inflammation, treating or reducing the inflammation may comprise treating or reducing rhinorrhea, sneezing, obstruction, and/or pain associated with the nasal inflammation. In another example, where the eosinophil-related inflammation is ocular inflammation, treating or reducing the inflammation may comprise treating or reducing erythema, swelling, pruritus, and difficulty seeing (i.e., loss of function) associated with the ocular inflammation. Treatment or reduction of eosinophil-related inflammation for additional tissues may be defined in a consistent manner based on the manner in which eosinophil-related inflammation manifests in the particular tissue as would be understood by a person having an ordinary level of skill in the art.

In some embodiments, the acidic amino acid is aspartate. In some embodiments, the polymer of aspartate is α poly-L-aspartic acid. In some embodiments, the acidic amino acid is glutamate. In some embodiments, the polymer of glutamate is selected from the group consisting of α poly-L-glutamic acid, α poly-D-glutamic acid, γ polyglutamic acid, and combinations thereof. However, it should be understood that additional types of acidic amino acids may be utilized herein. Furthermore, polymers of additional acidic anions may also be utilized herein for inhibiting the activity of eosinophil granule proteins without departing from the scope of this disclosure. For example, carboxymethylcellulose may be utilized in a similar manner to polyaspartic acid and polyglutamic acid as further described herein. In another example, polycarboxylic acid, polysulfonic acid, polyphosphoric acid, and/or polyboronic acid may be utilized in a similar manner to polyaspartic acid and polyglutamic acid as further described herein.

It should also be understood that additional variants of the amino acids and/or acidic substances may be utilized herein as would be known to a person having an ordinary level of skill in the art may be utilized herein. For example, while specific variants (e.g., α poly-L-aspartic acid, α poly-L-glutamic acid, α poly-D-glutamic acid, and/or γ polyglutamic acid) are particularly described herein, additional variants of the acidic compounds may be utilized to form the polymer as would be known to a person having an ordinary level of skill in the art. For example, different stereoisomers, variations on core structural functional groups and/or structures (e.g., α, β, γ, δ), and/or variants on the polarity, ionization, and/or side chain group type of the acidic compounds may be utilized herein. It should be understood that the potency of a particular acidic compound or a particular variant thereof may be based on the pKₐ of the acidic compounds. pKₐ is defined as:

$${pK}_{a} = - log ( {K}_{a} )$$

where Kₐ is the acid dissociation constant of the acidic compounds. Generally, the potency of a particular acidic compound or a particular variant thereof may increase as the pKₐ increases. For example, the pKₐ of aspartate is higher than the pKₐ of glutamate, thereby indicating that aspartate is a more potent acid and a more potent inhibitor of eMBP1. Accordingly, acidic compounds having similar pKₐ as aspartate and/or glutamate may be sufficiently potent acids to serve as inhibitors of eMBP1 as described herein. Furthermore, variants and/or modified version of a particular acidic compound may be selected to improve and/or optimize pKₐ, thereby improving and/or optimizing potency for inhibition of eMBP1.

In some instances, the polymers of the composition may be defined by a minimum pKₐ. For example, the polymers of the composition have a pKₐ of at least the pKₐ of glutamate. The pKₐ of glutamate may be described as about 2.1. In another example, the polymers of the composition have pKₐ of at least the pKₐ of aspartate. The pKₐ of aspartate may be described as about 1.92. However, it should be understood that the pKₐ attributed to an acid may vary as would be known to a person having an ordinary level of skill in the art. For example, the pKₐ may change due to certain modifications to the acid. In another example, the pKₐ may be different for a particular subtype of the acid (e.g., any of the subtypes of glutamate and aspartate as described herein). Accordingly, where a specific embodiment of the acid (e.g., a modified version and/or a subtype) are contemplated, the minimum pKₐ defining the composition may be based on the pKₐ of the specific embodiment of the acid, which could be ascertained by a person having an ordinary level of skill in the art by known methods. In some embodiments, the polymers of the composition have a pKₐ of at least about 1. In some embodiments, the polymers of the composition have a pKₐ of at least about 2. In some embodiments, the polymers of the composition have a pKₐ of at least about 3. In some embodiments, the polymers of the composition have a pKₐ of at least about 4. In some embodiments, the polymers of the composition have a pKₐ of at least about 5. In some embodiments, the polymers of the composition have a pKₐ of at least about 6. In some embodiments, the polymers of the composition have a pKₐ of at least about 7. In some embodiments, the polymers of the composition have a pKₐ of at least about 8. In some embodiments, the polymers of the composition have a pKₐ of at least about 9. In some embodiments, the polymers of the composition have a pKₐ of at least about 10. In some embodiments, the polymers of the composition have a pKₐ of at least greater than 10. In some embodiments, the polymers of the composition have a pKₐ of about 1 to about 15. In some embodiments, the polymers of the composition have a pKₐ of about 1 to about 20. In some embodiments, the polymers of the composition have a pKₐ of about 1 to about 10. In some embodiments, the polymers of the composition have a pKₐ of about 2 to about 10. In some embodiments, the polymers of the composition have a pKₐ of about 3 to about 10. In some embodiments, the polymers of the composition have a pKₐ of about 4 to about 10. In some embodiments, the polymers of the composition have a pKₐ of about 5 to about 10. In some embodiments, the polymers of the composition have a pKₐ of about 6 to about 10. In some embodiments, the polymers of the composition have a pKₐ of about 7 to about 10. In some embodiments, the polymers of the composition have a pKₐ of about 8 to about 10. In some embodiments, the polymers of the composition have a pKₐ of about 9 to about 10. In some embodiments, the polymers of the composition have a pKₐ of about 10 to about 15. In some embodiments, the polymers of the composition have a pKₐ of about 10 to about 20. In some embodiments, the polymers of the composition have a pKₐ of about 15 to about 20. In some embodiments, the polymers of the composition have a pKₐ of about 5 to about 15. In some embodiments, the polymers of the composition have a pKₐ of about 5 to about 20.

In some instances, the polymers of the composition may be defined by a minimum inhibitory activity for inhibiting EGPs (e.g., eMBP1) at a substantial rate. In some embodiments, the minimum inhibitory activity may be expressed as a concentration, i.e., a minimum inhibitory concentration (MIC) for inhibiting the activity of eMBP1. As further described in Example 2 and shown in Table 2, the half-maximal inhibitory concentration (IC50) expressed in molar concentration may be a useful indicator of the potency of a polymer for inhibiting the activity of eMBP1. Accordingly, the MIC may be expressed as an IC50 (M) value for the polymer. It should be understood that the MIC expressed as an IC50 (M) value is a threshold measure for the polymer that describes the potency of the polymer for inhibiting the EGP. When describing a class of polymers by MIC, it should be understood that the class would include polymers having an MIC at or below the threshold MIC. In some embodiments, the polymers of the composition have an IC50 (M) of about 2×10⁻⁶ or less (i.e., about the IC50 (M) of the WT Pro-Piece and/or low molecular weight heparin as shown in Table 2). In some embodiments, the polymers of the composition exhibit an IC50 (M) of about 1×10⁻⁶ or less. In some embodiments, the polymers of the composition exhibit an IC50 (M) of about 5×10⁻⁷ or less (i.e., about the IC50 (M) of unfractionated heparin). In some embodiments, the polymers of the composition exhibit an IC50 (M) of about 3×10⁻⁷ or less (i.e., about the IC50 (M) of high molecular weight heparin). In some embodiments, the polymers of the composition exhibit an IC50 (M) of about 1×10⁻⁷ or less. In some embodiments, the polymers of the composition exhibit an IC50 (M) of about 5×10⁻⁸ or less. In some embodiments, the polymers of the composition exhibit an IC50 (M) of about 1×10⁻⁸ or less. In some embodiments, the polymers of the composition exhibit an IC50 (M) of about 5×10⁻⁹ or less. In some embodiments, the polymers of the composition exhibit an IC50 (M) of about 1×10⁻⁹ or less.

In some embodiments described herein, the one or more eosinophil granule proteins comprise eosinophil major basic protein 1 (eMBP1). The acidic polymers described herein may be configured to bind to eMBP1, thereby inhibiting the activity of eMBP1 and neutralizing the toxic effects thereof on the tissue. However, additional eosinophil granule proteins may also cause inflammation in the tissue and may likewise be inhibited and neutralized by binding to the acidic polymers. Accordingly, in some embodiments, the one or more eosinophil granule proteins further comprise EDN, eMBP2, ECP, and/or EPO.

In some embodiments, the polymer has an average molecular weight of at least about 5 kDa. However, in additional embodiments, the polymer has an average molecular weight of at least about 10 kDa, about 20 kDa, about 30 kDa, about 40 kDa, about 50 kDa, about 100 kDa, about 200 kDa, about 300 kDa, about 400 kDa, about 500 kDa, about 600 kDa, about 700 kDa, about 800 kDa, about 900 kDa, about 1000 kDa, or individual values or ranges therebetween.

In some embodiments, the polymer has an average molecular weight of about 5 kDa to about 1000 kDa. In some embodiments, the polymer has an average molecular weight of about 5 kDa to about 700 kDa. In some embodiments, the polymer has an average molecular weight of about 5 kDa to about 500 kDa. In some embodiments, the polymer has an average molecular weight of about 5 kDa to about 250 kDa. In some embodiments, the polymer has an average molecular weight of about 5 kDa to about 100 kDa. In some embodiments, the polymer has an average molecular weight of about 5 kDa to about 50 kDa. In some embodiments, the polymer has an average molecular weight of about 5 kDa to about 20 kDa. In some embodiments, the polymer has an average molecular weight of about 5 kDa to about 10 kDa.

In some embodiments, the polymer has an average molecular weight of about 700 kDa to about 1000 kDa. In some embodiments, the polymer has an average molecular weight of about 500 kDa to about 1000 kDa. In some embodiments, the polymer has an average molecular weight of about 250 kDa to about 1000 kDa. In some embodiments, the polymer has an average molecular weight of about 100 kDa to about 1000 kDa. In some embodiments, the polymer has an average molecular weight of about 50 kDa to about 1000 kDa. In some embodiments, the polymer has an average molecular weight of about 20 kDa to about 1000 kDa. In some embodiments, the polymer has an average molecular weight of about 10 kDa to about 1000 kDa.

In additional embodiments, the polymer has an average molecular weight below about 5 kDa. For example, the polymer may have an average molecular weight of at least about 1 kDa, about 2 kDa, about 3 kDa, about 4 kDa, or individual values or ranges therebetween.

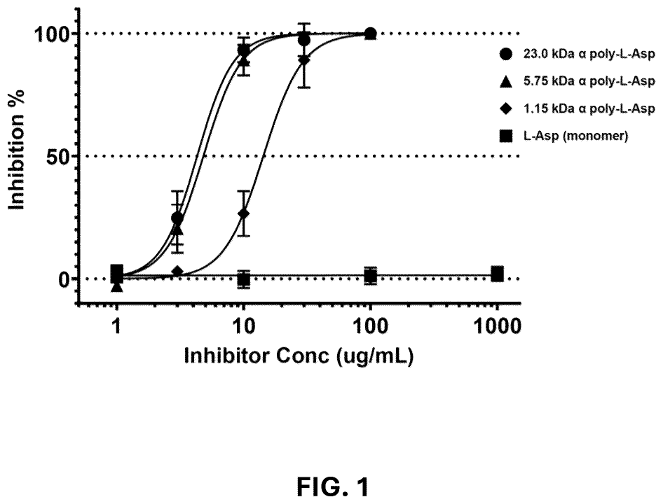

In some embodiments, the polymer may be polyglutamic acid and the average molecular weight of the polymer in the composition may be about 1.2 kDa, about 2.6 kDa, about 7.5 kDa, about 9 kDa, about 33 kDa, about 120 kDa, about 700 kDa, greater than about 700 kDa, or individual values or ranges therebetween. In a particular example, the polymer is α poly-L-glutamic acid and/or α poly-D-glutamic acid, and the average molecular weight of the polymer in the composition is about 2.6 kDa, about 7.5 kDa, about 9 kDa, about 33 kDa, about 120 kDa, or individual values or ranges therebetween. In another particular example, the polymer is γ polyglutamic acid and the average molecular weight of the polymer in the composition is about 1.2 kDa, less than about 10 kDa, about 10 kDa, about 700 kDa, greater than about 700 kDa, or individual values or ranges therebetween. In additional embodiments, the polymer is α poly-L-aspartic acid and the average molecular weight of the polymer in the composition is about 1.15 kDa, about 5.75 kDa, about 23 kDa, or individual values or ranges therebetween. Potencies for neutralization of eMBP1 are summarized in Table 2. Turning to FIG. 1, the potencies of several forms of aspartate for inhibiting eMBP1 is depicted in accordance with an embodiment. As shown, monomer aspartic acid was devoid of eMBP1 inhibitory activity, and the inhibitory activity of polyaspartic acid improved as molecular weight increased.

The average molecular weight of the polymer may be selected to optimize binding to sites expressing eosinophilic inflammation. Because the polymer exhibits a higher affinity for eMBP1 and/or other eosinophil granule proteins than relatively lower molecular weight forms thereof, the polymer will bind more avidly to sites of eosinophilic inflammation than the relatively lower molecular weight forms. In some embodiments, binding affinity of the polymer may increase as a function of its molecular weight. However, the binding affinity may also plateau, e.g., the relative increase in binding affinity may diminish as molecular weight increases (see FIG. 1 as further described herein). Accordingly, as the average molecular weight of the polymer increases, the quantity of polymer required for localization of eosinophilic inflammation can be reduced with the expectation that a greater percentage of administered polymer will localize to the inflammation sites.

The purity of the HMW polymer can be defined by the amount of chains of the polymer having a molecular weight above a predetermined threshold. For example, the predetermined threshold may be about 5 kDa and accordingly the purity of the HMWH can be determined based on a fraction, percentage, or ratio of chains of the polymer in the composition having a molecular weight of at least about 5 kDa compared to those having a molecular weight of less than about 5 kDa. In some embodiments, at least about 50% of the chains of the polymer in the composition have a molecular weight of about 5 kDa or greater, which may also be referred to as a purity of about 50% (i.e., “high purity”). In some embodiments, the total percentage of chains of the polymer in the composition having a molecular weight of about 5 kDa or greater may be about 60%, about 70%, about 80%, about 90%, about 95%, about 100%, or individual values or ranges therebetween. Accordingly, the composition may be described as having about 60% purity, about 70% purity, about 80% purity, about 90% purity, about 95% purity, about 100% purity, or individual values or ranges therebetween. In some embodiments, the polymer can also be defined by a maximum amount of molecular chains with a molecular weight below the predetermined threshold. For example, the composition can comprise a percentage of chains of the polymer with a molecular weight below 5 kDa at or below about 50%, about 40%, about 30%, about 25%, about 20%, about 15%, about 10%, about 5%, about 0%, or individual values or ranges therebetween.

In some embodiments, a composition with a relatively high purity (e.g., 80%) of the polymer may demonstrate greater localization to the eosinophil-related inflammation site than a composition with a lower purity (e.g., 50%). In some embodiments, the localization rate of the composition increases as the purity of the polymer increases. Accordingly, as the purity of the composition increases, the quantity of polymer required for adequate localization of eosinophilic inflammation can be reduced with the expectation that a greater percentage of administered polymer will localize to the inflammation sites.

In some embodiments, the predetermined threshold for molecular weight that is used to define the “purity” of the composition can be a value other than 5 kDa. The predetermined threshold can be set based on the minimum desired average molecular weight for the polymer in the composition. For example, the predetermined threshold for assessing purity of the HMWH can be about 1 kDa, about 2 kDa, about 3 kDa, about 4 kDa, about 10 kDa, about 20 kDa, about 30 kDa, about 40 kDa, about 50 kDa, about 100 kDa, about 200 kDa, about 300 kDa, about 400 kDa, about 500 kDa, about 600 kDa, about 700 kDa, about 800 kDa, about 900 kDa, about 1000 kDa, or individual values or ranges therebetween. Similarly, the cutoff of the low molecular weight chains can be a value other than 8 kDa. For example, the cutoff may be 5 kDa, 6 kDa, 7 kDa, 8 kDa, 9 kDa, 10 kDa, 11 kDa, 12 kDa, greater than 12 kDa, or individual values or ranges therebetween.

The compositions disclosed herein may comprise a specified quantity of the acidic polymer. In some embodiments, the specified quantity of the acidic polymer is a dose of the acidic polymer configured to reach or localize to an eosinophil-related inflammation site. In some embodiments, the specified quantity of the acidic polymer is a therapeutically effective amount of the acidic polymer. In some embodiments, the specified quantity of the acidic polymer is a dose of the acidic polymer configured to localize to the eosinophil-related inflammation site and facilitate imaging and/or diagnosis thereof. For example, where the eosinophil-related inflammation site is an esophagus or portion of an esophagus, the composition may comprise a quantity of the acidic polymer selected from about 15000 units, about 10000 units, about 5000 units, about 4000 units, about 3000 units, about 2000 units, about 1000 units, about 500 units, about 250 units, less than about 250 units, or individual values or ranges therebetween. The quantity of the acidic polymer may be about 100 mg, about 90 mg, about 80 mg, about 70 mg, about 60 mg, about 50 mg, about 40 mg, about 30 mg, about 20 mg, about 10 mg, about 5 mg, about 4 mg, about 3 mg, about 2 mg, about 1 mg, about 0.5 mg, less than about 0.5 mg, or individual values or ranges therebetween. In some embodiments, the quantity of the acidic polymer is diluted (e.g., with sterile saline) to provide a final volume of about 15 mL, about 14 mL, about 13 mL, about 12 mL, about 11 mL, about 10 mL, about 9 mL, about 8 mL, about 7 mL, about 6 mL, about 5 mL, about 4 mL, about 3 mL, about 2 mL, about 1 mL, about 0.9 mL, about 0.8 mL, about 0.7 mL, about 0.6 mL, about 0.5 mL, about 0.4 mL, about 0.3 mL, about 0.2 mL, about 0.1 mL, less than about 0.1 mL, or individual values or ranges therebetween. The dose of the acidic polymer may vary based on the size of the targeted eosinophil-related inflammation site. A larger quantity of the acidic polymer may be required for targeting larger sites and/or organs. Where the eosinophil-related inflammation site is a different site or organ other than the esophagus as further described herein, the quantity of the acidic polymer may be a value described herein or a larger or small value necessary to adequately target the eosinophil-related inflammation site as would be apparent to one having an ordinary level of skill in the art.

In some embodiments, the therapeutically effective dose is about 50 mg. In some embodiments, the therapeutically effective dose is about 100 mg. In some embodiments, the therapeutically effective dose is about 150 mg. In some embodiments, the therapeutically effective dose is about 200 mg. In some embodiments, the therapeutically effective dose is about 250 mg. In some embodiments, the therapeutically effective dose is about 500 mg. In some embodiments, the therapeutically effective dose is about 1000 mg. However, the therapeutically effective dose may be tailored based on a variety of factors as would be known to a person having an ordinary level of skill in the art to enable sufficient inhibition of the eosinophil granule proteins at the tissue site by the EGP inhibitor.

In some embodiments, the compositions disclosed herein are administered orally. For example, the composition may be swallowed orally by the subject. In some embodiments, the composition can be administered orally with a syringe, dropper, or other device. In some embodiments, the compositions disclosed herein may be administered orally or topically as an oral or topical solution. For example, compositions comprising the acidic polymer may be formulated as an oral solution or a topical solution for treating eosinophil-related GI disorders including by not limited to eosinophilic esophagitis, eosinophilic gastroenteritis, and inflammatory bowel disease including but not limited to ulcerative colitis and Crohn's disease. In some embodiments, the compositions disclosed herein may be administered by inhalation as a nasal spray. For example, compositions comprising the acidic polymer may be formulated as a nasal spray for treating eosinophilic chronic rhinosinusitis or nasal polyps. In some embodiments, the compositions disclosed herein may be administered topically (e.g., as eye drops). For example, compositions comprising the acidic polymer may be formulated for topical administration for treating ocular diseases having an allergic pathophysiological component including but not limited to eosinophilic conjunctivitis, seasonal and/or perennial allergic conjunctivitis, vernal conjunctivitis, atopic keratoconjunctivitis, giant papillary conjunctivitis or contact dermatoconjunctivitis.

The compositions disclosed herein may be configured or formulated for additional administration routes. In some embodiments, the compositions disclosed herein are configured for administration systemically and/or intravenously to treat gastrointestinal eosinophil-associated diseases. This is in contrast to other known EGP inhibitors, which are not safe for systemic and/or intravenous administration due to complications and side effects (e.g., heparin-induced thrombocytopenia). In additional embodiments, the compositions disclosed herein are configured for administration systemically, intravenously, topically, by inhalation and/or orally to treat gastrointestinal eosinophil-associated diseases. In some embodiments, the gastrointestinal eosinophil-associated diseases that may be treated by oral (or topical) administration comprise eosinophilic esophagitis, eosinophilic gastritis, and/or eosinophilic gastroenteritis. In some embodiments, the compositions disclosed herein are configured for administration by inhalation to treat inflammation in the nose, paranasal sinuses, and lung. In some embodiments, the compositions disclosed herein are configured for administration by an enema to treat the colon. In some embodiments, the compositions disclosed herein are configured for administration by catheter to treat eosinophil-related inflammation in the urinary bladder. In some embodiments, the compositions disclosed herein are configured for administration by eye drops to treat ocular eosinophil-related inflammation or diseases having an allergic pathophysiological component. In some embodiments, the compositions disclosed herein are configured for topical administration as a cream or ointment to treat eosinophil-related inflammation and/or diseases of the skin. The compositions described herein may be administered in the conventional manner by any route where they are active and effective for localizing to a tissue exhibiting eosinophil-related inflammation. For example, administration can be systemic, topical, intra-orificial, and/or via a medical tube or medical device. For example, administration can be, but is not limited to, parenteral, intravenous, intramuscular, intraperitoneal, transdermal, intravaginally, and the like. Thus, modes of administration (either alone or in combination with other pharmaceuticals) can be, but are not limited to, injectable (including short-acting, depot, implant and pellet forms injected subcutaneously or intramuscularly), topical (including ointments or creams, e.g., for application to the skin and/or within an orifice), and/or by use of vaginal creams, suppositories, pessaries, vaginal rings, reservoirs, and transdermal forms such as patches and creams. Specific modes of administration may depend on the location of the site of eosinophil-related inflammation.

The composition may be configured for administration to various tissues or organs. In some embodiments, the targeted eosinophil-related inflammation or eosinophilic disease may be specific to the gastrointestinal tract (e.g., mouth, esophagus, stomach, small intestine, large intestine, or colon), lung, nose, eye, skin, one or more joints, one or more muscles, one or more nerves, heart, kidney, bladder, uterus, prostate, breast, lymph or blood.

In some embodiments, the compositions may further comprise one or more additional agents. In some embodiments, the composition further comprises a therapeutic agent conjugated to the acidic polymer. In some embodiments, the composition further comprises a therapeutically effective amount of a therapeutic agent for administration to the patient. In some embodiments, the therapeutic agent is configured or formulated to have a therapeutic effect on the eosinophil-related inflammation and/or disease. As disclosed herein, by conjugating therapeutic agents to the acidic polymer, a treatment can be targeted directly to an area(s) of inflammation because the avidity of the acidic polymer for tissue-bound EGPs. Thus, the targeting of the acidic polymer conjugated to a therapeutic agent (e.g., an acidic polymer/therapeutic agent complex) directly to one or more sites of eosinophil-related inflammation can reduce the quantity (or dose) of the therapeutic agent needed for care, and thus limit or minimize any side effects associated with the administration of the therapeutic agent. Accordingly, the therapeutically effective amount of the therapeutic agent can be less than a therapeutically effective amount typically associated with administration of the therapeutic agent in the absence of the acidic polymer or another targeted mechanism. In some embodiments, the therapeutic agent is a glucocorticoid, which is an effective treatment for eosinophil-related diseases. In some embodiments, the glucocorticoid is one or more of mometasone, fluticasone, budesonide, and methylprednisolone. Additional therapeutic agents for eosinophil-related inflammation or diseases are contemplated as would be apparent to one having an ordinary level of skill in the art.

In additional embodiments, the composition may further comprise a tracer such as a radiolabeled contrast agent conjugated to the acidic polymer in order to enable imaging and/or diagnosis of eosinophil-related conditions. Accordingly, embodiments herein are also directed to methods of producing a medical image of an organ in a subject. The method comprises administering, to the subject, a composition comprising an effective dose of a radiolabeled polymer of an acidic amino acid or a pharmaceutically acceptable salt thereof and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and detecting the radiolabeled polymer/eosinophil granule protein complex in a mucosal tissue of the organ, thereby producing a medical image of the organ. Furthermore, embodiments herein are also directed to methods of diagnosing eosinophil-related inflammation in an organ of a subject. The method comprises administering, to the subject, a composition comprising an effective dose of a radiolabeled polymer of an acidic amino acid or a pharmaceutically acceptable salt thereof and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and detecting the radiolabeled polymer/eosinophil granule protein complex in a mucosal tissue of the organ, thereby diagnosing the eosinophil-related inflammation in the organ. In some embodiments, the radiolabeled polymer/eosinophil granule protein complex is detected using single-photon emission computed tomography (SPECT), positron emission tomography (PET), conventional or computed tomography (CT), magnetic resonance imaging (MRI), or a combination thereof.

In some embodiments, the radiolabeled polymer comprises the composition as described herein and further comprising a tracer such as a radiolabeled contrast agent conjugated to the acidic polymer. For example, the radiolabeled contrast agent can be ^{99m}Tc. However, other tracers, such as tracers used for positron emission tomography, may also be employed for detecting the binding of the acidic polymer to sites of eosinophilic inflammation. In some embodiments, the tracer may be any tracer or label as described in Table 1. Accordingly, when the composition is administered as described herein, conventional imaging modalities may be used to visualize the eosinophil-related inflammation and/or disease, e.g., single-photon emission computed tomography (SPECT), positron emission tomography (PET), conventional or computed tomography (CT), magnetic resonance imaging (MRI), or a combination thereof. For example, in the case of eosinophilic esophagitis, the composition may be administered to facilitate visualization of the entire esophagus.

In some embodiments, the composition comprising a tracer may be used to diagnose eosinophil-related inflammation and/or disease. For example, the composition comprising a tracer (i.e., a diagnostic agent) may be administered as described herein and conventional imaging modalities may be used to capture one or more images of the patient. Localization of the acidic polymer may be assessed based on the location and concentration of the detected tracer in the one or more images. Accordingly, the patient may be diagnosed with eosinophil-related inflammation and/or disease based on the one or more images. In some embodiments, at least one first image of the patient is acquired at a first time and at least one second image of the patient is acquired at a second time. The first image and the second image may be compared to monitor and assess progression of the inflammation and/or disease activity. In some embodiments, additional images may be acquired at additional times to continue to monitor and assess the patient. In some embodiments, a separate administration of the composition may occur prior to acquiring each of the first image, the second image, and any of the additional images. However, in some embodiments, a single administration of the composition may provide adequate radiolabeling for more than one set of images. The composition may be utilized for monitoring and assessing any of the eosinophil-related conditions and diseases described herein with respect to treatment.

The radiolabeled polymer disclosed herein can be prepared at various doses. For example, the radiolabeled polymer can be about 0.1, about 0.2, about 0.3, about 0.4, about 0.5, about 0.6, about 0.7, about 0.8, about 0.9, about 1.0, about 1.5, about 2.0, about 2.5, about 3, about 3.5, about 4.0, about 4.5, about 5.0, about 5.5, about 6.0, about 6.5, about 7.0, about 7.5, about 8.0, about 8.5, about 9.0, about 9.5, or about 10.0 mCi. In some embodiments, the dose of radiolabeled polymer may be about 0.3 mCi to about 1 mCi. In some embodiments, the dose of radiolabeled polymer may be about 1.0 mCi. In some embodiments, the dose of radiolabeled polymer may be about 10 mCi.

In some embodiments, the dose of polymer used to bind to ^{99m}Tc may change or vary depending on the avidity of the polymer for EGPs. In some embodiments, using high molecular weight polymers allows for the use of smaller amounts or doses of a radiolabel because it results in a higher rate of binding at the site of eosinophil-related inflammation compared to other EGP inhibitors. Accordingly, the dose or amount of the polymer can be less than required with conventional EGP inhibitors. For example, the amount of polymer may be about 0.1 to about 1 mg, about 1 mg to about 2 mg, or about 2 mg to about 3 mg, or greater than about 3 mg in order to provide an effective amount of the radiolabel.

The compositions disclosed herein can further comprise various additional components or additives as would be known to a person having an ordinary level of skill in the art. In some embodiments, the compositions further comprise stannous chloride. In some embodiments, the compositions further comprise a stabilizing agent. In some embodiments, the compositions further comprise a taste-masking agent. In some embodiments, a radiolabeled contrast agent may be suspended in a thickened mixture (i.e., sucralose). Examples of thickening agents include, but are not limited to, dietary starches, such as agar-agar, alginate, carrageenan, cassia gum, cellulose gum, gellan gum, guar gum, hydroxypropylcellulose, konjac gum, locust bean gum, methylcellulose, hydroxypropyl methylcellulose, microcrystalline cellulose, pectin, and xanthan gum. Other viscosifiers include honey, agave nectar, date nectar, Kuzu or Kudzu root, arrow root, corn syrup, thick juices, maple syrup, coconut oil, and palm oil.

The methods as described herein are not intended to be limited in terms of the particular embodiments described, which are intended only as illustrations of various features. Many modifications, variations, and additions to the methods can be made without departing from their spirit and scope, as will be apparent to those skilled in the art.

While various acidic polymers are disclosed herein, it should be understood that a plurality of acidic polymers may be utilized in combination at varying dosages in order to obtain an additive and/or synergistic effect on the inhibition of EGPs. For example, in some embodiments, the acidic polymer may comprise glutamate and aspartate polymerized together, i.e., a combination polymer or co-polymer. In some embodiments, the glutamate and aspartate may be provided in the polymer at a ratio of about 1:1. In additional embodiments, the glutamate and aspartate may be provided in the polymer at a ratio of about 1:2, about 1:3, about 1:4, about 1:5, about 1:6, about 1:7, about 1:8, about 1:9, about 1:10, about 1:20, about 1:30, about 1:40, about 1:50, about 1:100, or individual ratios or ranges therebetween. In additional embodiments, the glutamate and aspartate may be provided in the polymer at a ratio of about 2:1, about 3:1, about 4:1, about 5:1, about 6:1, about 7:1, about 8:1, about 9:1, about 10:1, about 20:1, about 30:1, about 40:1, about 50:1, about 100:1, or individual ratios or ranges therebetween. In additional embodiments, the composition may comprise a combination of polyglutamic acid and polyaspartic acid as described herein. The polyglutamic acid and polyaspartic acid may be provided within the polymer in any of the above-listed ratios.

In some embodiments, an EGP inhibitor may be combined with a pharmaceutically acceptable excipient to produce a composition for administration to a subject as described herein.

According to some embodiments, a method of treating a tissue exhibiting eosinophil-related inflammation in a subject is provided. The method comprising administering to the subject a composition comprising: a therapeutically effective amount of a polymer of an acidic amino acid or a pharmaceutically acceptable salt thereof, wherein the polymer has an average molecular weight of at least about 5 kDa; and a pharmaceutically acceptable excipient, wherein the polymer binds to one or more eosinophil granule proteins in the tissue to treat the eosinophil-related inflammation.

According to some embodiments, a method of reducing eosinophil-related inflammation in a tissue in a subject, the method comprising administering to the subject a composition comprising: a therapeutically effective amount of a polymer of an acidic amino acid or a pharmaceutically acceptable salt thereof, wherein the polymer has an average molecular weight of at least about 5 kDa; and a pharmaceutically acceptable excipient, wherein the polymer binds to one or more eosinophil granule proteins in the tissue to reduce the eosinophil-related inflammation.

According to some embodiments, the acidic amino acid is selected from the group consisting of aspartate, glutamate, and combinations thereof.

According to additional embodiments, the polymer of glutamate is selected from the group consisting of α poly-L-glutamic acid, α poly-D-glutamic acid, γ polyglutamic acid, and combinations thereof. According to further embodiments, the average molecular weight of the polymer of α poly-L-glutamic acid is selected from the group consisting of: about 5 kDa to about 7.5 kDa, about 7.5 kDa to about 9 kDa, about 9 kDa to about 33 kDa, about 33 kDa to about 120 kDa, and greater than about 120 kDa. According to further embodiments, the average molecular weight of the polymer of α poly-D-glutamic acid is selected from the group consisting of: about 5 kDa to about 33 kDa, and greater than about 33 kDa. According to further embodiments, the average molecular weight of the polymer of γ polyglutamic acid is selected from the group consisting of: about 5 kDa to about 10 kDa, about 10 kDa to about 50 kDa, about 50 kDa to about 100 kDa, about 100 kDa to about 500 kDa, about 500 kDa to about 700 kDa, and greater than about 700 kDa.

According to additional embodiments, the polymer of aspartate is α poly-L-aspartic acid. According to further embodiments, the average molecular weight of the polymer of α poly-L-aspartic acid is selected from the group consisting of: about 5 kDa to about 5.75 kDa, about 5.75 kDa to about 23 kDa, and greater than about 23 kDa.

According to some embodiments, the average molecular weight of the polymer is at least about 10 kDa. According to additional embodiments, the average molecular weight of the polymer is at least about 20 kDa. According to further embodiments, the average molecular weight of the polymer is at least about 50 kDa. According to still further embodiments, the average molecular weight of the polymer is at least about 100 kDa. According to still further embodiments, the average molecular weight of the polymer is at least about 500 kDa. According to still further embodiments, the average molecular weight of the polymer is at least about 700 kDa.

According to some embodiments, the acidic amino acid is aspartate, wherein the average molecular weight of the polymer is at least about 20 kDa.

According to some embodiments, the acidic amino acid is glutamate, wherein the average molecular weight of the polymer is at least about 100 kDa. According to additional embodiments, the average molecular weight of the polymer is at least about 700 kDa.

According to some embodiments, the one or more eosinophil granule proteins comprise one or more of major basic protein 1 (eMBP1), major basic protein 2 (eMBP2), eosinophil derived neurotoxin (EDN), eosinophil cationic protein (ECP), and eosinophil peroxidase (EPO).

According to some embodiments, the method further comprises a therapeutic agent conjugated to the polymer. According to additional embodiments, the therapeutic agent is a glucocorticoid.

According to some embodiments, at least 50% of chains of the polymer in the composition have a molecular weight of at least 5 kDa. According to additional embodiments, at least 60% of chains of the polymer in the composition have a molecular weight of at least 5 kDa. According to further embodiments, at least 70% of chains of the polymer in the composition have a molecular weight of at least 5 kDa.

According to some embodiments, the composition is administered systemically.

According to some embodiments, a composition for use in treating a tissue exhibiting eosinophil-related inflammation in a subject is provided. The composition comprises: an effective amount of a polymer of an acidic amino acid or a pharmaceutically acceptable salt thereof, the polymer having an average molecular weight of at least about 5 kDa, wherein the polymer is configured to bind to one or more eosinophil granule proteins in the tissue to treat the eosinophil-related inflammation; and a pharmaceutically acceptable excipient.

According to some embodiments, the acidic amino acid is selected from the group consisting of aspartate, glutamate, and combinations thereof.

According to some embodiments, the polymer of glutamate is selected from the group consisting of α poly-L-glutamic acid, α poly-D-glutamic acid, γ polyglutamic acid, and combinations thereof. According to additional embodiments, the average molecular weight of the polymer of α poly-L-glutamic acid is selected from the group consisting of: about 5 kDa to about 7.5 kDa, about 7.5 kDa to about 9 kDa, about 9 kDa to about 33 kDa, about 33 kDa to about 120 kDa, and greater than about 120 kDa. According to additional embodiments, the average molecular weight of the polymer of α poly-D-glutamic acid is selected from the group consisting of: about 5 kDa to about 33 kDa, and greater than about 33 kDa. According to additional embodiments, the average molecular weight of the polymer of γ polyglutamic acid is selected from the group consisting of: about 5 kDa to about 10 kDa, about 10 kDa to about 50 kDa, about 50 kDa to about 100 kDa, about 100 kDa to about 500 kDa, about 500 kDa to about 700 kDa, and greater than about 700 kDa.

According to some embodiments, the polymer of aspartate is α poly-L-aspartic acid. According to additional embodiments, the average molecular weight of the polymer of a poly-L-aspartic acid is selected from the group consisting of: about 5 kDa to about 5.75 kDa, about 5.75 kDa to about 23 kDa, and greater than about 23 kDa.

According to some embodiments, the average molecular weight of the polymer is at least about 10 kDa. According to additional embodiments, the average molecular weight of the polymer is at least about 20 kDa. According to further embodiments, the average molecular weight of the polymer is at least about 50 kDa. According to still further embodiments, the average molecular weight of the polymer is at least about 100 kDa. According to still further embodiments, the average molecular weight of the polymer is at least about 500 kDa. According to still further embodiments, the average molecular weight of the polymer is at least about 700 kDa.

According to some embodiments, the acidic amino acid is aspartate, wherein the average molecular weight of the polymer is at least about 20 kDa.

According to some embodiments, the acidic amino acid is glutamate, wherein the average molecular weight of the polymer is at least about 100 kDa. According to additional embodiments, the average molecular weight of the polymer is at least about 700 kDa.

According to some embodiments, the one or more eosinophil granule proteins comprise one or more of major basic protein 1 (eMBP1), major basic protein 2 (eMBP2), eosinophil derived neurotoxin (EDN), eosinophil cationic protein (ECP), and eosinophil peroxidase (EPO).

According to some embodiments, the composition further comprises a therapeutic agent conjugated to the polymer. According to additional embodiments, the therapeutic agent is a glucocorticoid.

According to some embodiments, at least 50% of chains of the polymer in the composition have a molecular weight of at least 5 kDa. According to additional embodiments, at least 60% of chains of the polymer in the composition have a molecular weight of at least 5 kDa. According to further embodiments, at least 70% of chains of the polymer in the composition have a molecular weight of at least 5 kDa.

According to some embodiments, the composition is configured to be administered systemically.

According to some embodiments, the composition further comprises a radiolabeled contrast agent conjugated to the polymer. According to additional embodiments, the radiolabeled contrast agent is ^{99m}Tc.

According to some embodiments, a method of producing a medical image of an organ in a subject is provided. The method comprises: administering, to the subject, a composition comprising an effective dose of a radiolabeled polymer of an acidic amino acid or a pharmaceutically acceptable salt thereof and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and detecting the radiolabeled polymer/eosinophil granule protein complex in a mucosal tissue of the organ, thereby producing a medical image of the organ.

According to some embodiments, a method of diagnosing eosinophil-related inflammation in an organ of a subject is provided. The method comprises: administering, to the subject, a composition comprising an effective dose of a radiolabeled polymer of an acidic amino acid or a pharmaceutically acceptable salt thereof and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and detecting the radiolabeled polymer/eosinophil granule protein complex in a mucosal tissue of the organ, thereby diagnosing the eosinophil-related inflammation in the organ.

According to some embodiments, the radiolabeled polymer/eosinophil granule protein complex is detected using single-photon emission computed tomography (SPECT), positron emission tomography (PET), conventional or computed tomography (CT), magnetic resonance imaging (MRI), or a combination thereof.

According to some embodiments, the acidic amino acid is selected from the group consisting of aspartate, glutamate, and combinations thereof.

According to some embodiments, the polymer of glutamate is selected from the group consisting of α poly-L-glutamic acid, α poly-D-glutamic acid, γ polyglutamic acid, and combinations thereof. According to additional embodiments, the average molecular weight of the polymer of α poly-L-glutamic acid is selected from the group consisting of: about 5 kDa to about 7.5 kDa, about 7.5 kDa to about 9 kDa, about 9 kDa to about 33 kDa, about 33 kDa to about 120 kDa, and greater than about 120 kDa. According to additional embodiments, the average molecular weight of the polymer of α poly-D-glutamic acid is selected from the group consisting of: about 5 kDa to about 33 kDa, and greater than about 33 kDa. According to additional embodiments, the average molecular weight of the polymer of γ polyglutamic acid is selected from the group consisting of: about 5 kDa to about 10 kDa, about 10 kDa to about 50 kDa, about 50 kDa to about 100 kDa, about 100 kDa to about 500 kDa, about 500 kDa to about 700 kDa, and greater than about 700 kDa.

According to some embodiments, the polymer of aspartate is α poly-L-aspartic acid. According to additional embodiments, the average molecular weight of the polymer of a poly-L-aspartic acid is selected from the group consisting of: about 5 kDa to about 5.75 kDa, about 5.75 kDa to about 23 kDa, and greater than about 23 kDa.

According to some embodiments, the average molecular weight of the polymer is at least about 10 kDa. According to additional embodiments, the average molecular weight of the polymer is at least about 20 kDa. According to further embodiments, the average molecular weight of the polymer is at least about 50 kDa. According to still further embodiments, the average molecular weight of the polymer is at least about 100 kDa. According to still further embodiments, the average molecular weight of the polymer is at least about 500 kDa. According to still further embodiments, the average molecular weight of the polymer is at least about 700 kDa.

According to some embodiments, the acidic amino acid is aspartate, wherein the average molecular weight of the polymer is at least about 20 kDa.

According to some embodiments, the acidic amino acid is glutamate, wherein the average molecular weight of the polymer is at least about 100 kDa. According to additional embodiments, the average molecular weight of the polymer is at least about 700 kDa.

According to some embodiments, the one or more eosinophil granule proteins comprise one or more of major basic protein 1 (eMBP1), major basic protein 2 (eMBP2), eosinophil derived neurotoxin (EDN), eosinophil cationic protein (ECP), and eosinophil peroxidase (EPO).

According to some embodiments, the radiolabeled polymer comprises a radiolabeled contrast agent conjugated to a polymer. According to additional embodiments, the radiolabeled contrast agent is ^{99m}Tc.

According to some embodiments, the composition further comprises a therapeutic agent conjugated to the polymer. According to additional embodiments, the therapeutic agent is a glucocorticoid.

According to some embodiments, at least 50% of chains of the polymer in the composition have a molecular weight of at least 5 kDa. According to additional embodiments, at least 60% of chains of the polymer in the composition have a molecular weight of at least 5 kDa. According to further embodiments, at least 70% of chains of the polymer in the composition have a molecular weight of at least 5 kDa.

According to some embodiments, the composition is administered systemically.

The compositions and methods disclosed herein have at least three advantages. First, for the localization of eosinophil-related inflammation, high molecular weight acidic polymers will bind more avidly than low molecular weight polymers and/or other EGP inhibitors to sites of eosinophil-related inflammation. In turn, the quantity of the composition required for effective localization to eosinophil-related inflammation (e.g., for treatment, diagnosis, etc.) may be reduced with the expectation that a greater percentage of the composition will localize to the one or more sites of eosinophil-related inflammation. Furthermore, utilizing the compositions and methods disclosed herein for imaging and/or diagnosis may reduce the quantity of the composition (and thus the quantity of radioactivity) required for effectively localization to eosinophil-related inflammation, thereby limiting a patient's exposure to radioactivity. Still further, the compositions and methods described herein may be used to identify eosinophil-related inflammation in and throughout the body (e.g., any organ of the human body afflicted with eosinophil-related inflammation.

Second, compositions comprising high molecular weight acidic polymers as described herein may also be more effective for neutralizing the toxic effects of eMBP1 and other EGPs as compared to low molecular weight polymers and/or other EGP inhibitors. For example, the high molecular weight acidic polymers may meet a threshold minimum inhibitory concentration (MIC) such as a threshold half-maximal inhibitory concentration (IC50 (M)) value meeting or exceeding that of conventionally known EGP inhibitors, thereby indicating that the high molecular weight acidic polymers are as potent or more potent as EGP inhibitors than the conventionally known inhibitors. In another example, the high molecular weight acidic polymers may meet a threshold pKₐ meeting or exceeding that of conventionally known EGP inhibitors, thereby indicating that the high molecular weight acidic polymers are as potent or more potent as EGP inhibitors than the conventionally known inhibitors. In some embodiments, the high molecular weight acidic polymers have the capacity to function as a medication by application to or delivery to one or more sites of eosinophil-related inflammation. For example, compositions comprising high molecular weight acidic polymers as described herein may be used to treat eosinophil-related gastrointestinal tract diseases (e.g., eosinophilic esophagitis) by oral administration of the composition to neutralize eosinophil-related inflammation. Furthermore, because the composition may be used to target eosinophil-related inflammation and because glucocorticoids such as fluticasone or budesonide can be conjugated to the high molecular weight heparin, the glucocorticoids or other additional therapeutics can be more effectively targeted to sites of eosinophil-related inflammation for treatment thereof.

Third, the compositions comprising high molecular weight acidic polymers as described herein may also be configured to systemic administration and/or intravenous administration in a clinically safe manner. Some conventionally known EGP inhibitors are not safe for systemic and/or intravenous administration because of complications and/or side effects. For example, administering heparin systemically and/or intravenously may lead to dangerous conditions known as heparin-induced thrombocytopenia (HIT) and heparin-induced thrombocytopenia and thrombosis (HITT). While the risk of HIT and/or HITT is limited for low molecular weight heparin, the risk is higher when administering unfractionated heparin or high molecular weight heparin (i.e., the more potent EGP inhibitors). Accordingly, the disclosed compositions comprising high molecular weight acidic polymers provide a useful alternative EGP inhibitor that can safely be administered systemically and/or intravenously while having similar or greater inhibitory activity as unfractionated heparin and/or high molecular weight heparin. Furthermore, the ability to be administered systemically and/or intravenously enables the use of the compositions to target a large number of tissues, organs, and conditions as described herein that cannot be treated by the alternative administration routes available for conventionally known EGP inhibitors (e.g., topical, oral, etc.).

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples:

### EXAMPLES

Studies of eosinophils have led to the identification and study of eosinophil major basic protein (eMBP1), a remarkably active granule protein stored in the granule core of the eosinophil. eMBP1 is synthesized in a precursor form known as pro-eMBP1 and later converted to eMBP1 in the eosinophilic leukocyte. When stored in the granule core, eMBP1 is inactive. However, once released, eMBP1 is a potent cytotoxin and cytostimulant capable of activating cells and/or inflaming and damaging tissue. In the following examples, studies to explore the suggestion that binding an acidic polymer to eMBP1 neutralizes the toxic effects of eMBP1 on a tissue.

### Example 1: Investigation of eMBP1 Neutralization Potencies for Acidic Polymers

Methods: The goal of the investigation is to design and test a peptide product to neutralize and, thus, mitigate toxic effects of eosinophil granule proteins as a treatment for eosinophil-related diseases. Specifically, one aim is to test various polymers of acidic amino acids (i.e., glutamate and aspartate) for their ability to neutralize eMBP1 and compare their potencies to those of the pro-piece of pro-eosinophil major basic protein 1 (pro-eMBP1) as well as various heparins, i.e., low molecular weight heparin, high molecular weight heparin, and unfractionated heparin.

The eosinophil is a peripheral blood leukocyte containing an abundance of cytoplasmic granules, rich in cationic protein toxins. Among these, the most abundant on a molar basis is eMBP1, which is exceedingly cationic with a calculated pI of 11.4. eMBP1 is toxic to helminths, bacteria, and numerous cells, such as respiratory epithelium, and is stimulatory to other cells, including basophils and mast cells. Studies of human diseases show that eMBP1 is present in secretions from patients with eosinophil-related diseases, including asthma, chronic rhinosinusitis, and gastrointestinal diseases, and is deposited on damaged tissues. Numerous studies show that the eosinophil mediates its damage to parasites and tissues by depositing its toxin-rich granule proteins onto microbial targets and tissues. Therefore, neutralization of eMBP1 is proposed as a treatment to mitigate tissue damage in eosinophil-related inflammation. No current therapies are directed at neutralization of granule proteins. Sequencing of eMBP1 cDNA indicated that eMBP1 is synthesized as a precursor composed of eMBP1 with an exceedingly acidic pro-piece and referred to as pro-eMBP1. Developing eosinophils synthesize pro-eMBP1, and the pro-piece is removed during granule maturation. Studies of compounds mimicking pro-piece established that they can neutralize toxic effects of eMBP1 and, also, of another granule protein, eosinophil cationic protein (ECP). We have expressed pro-piece, found that it is glycan-rich, and demonstrated its binding to eMBP1 by surface plasmon resonance. In this project, we aim to determine the characteristics of pro-piece that maximally neutralize the toxic effects of eMBP1 with the goal of identifying compounds able to neutralize granule protein toxins as a therapy for eosinophil-related diseases.

Because pro-piece (PP) can neutralize the toxicity of eMBP1, we reasoned that a careful study of the carbohydrate and acidic peptide structures might elucidate the comparative functions of these domains and lead to an effective eMBP1 inhibitor. We obtained heparins and polymers of the acidic amino acids, glutamic and aspartic, and compared their potencies in the eMBP1 neutralization assay.

Results: The results are summarized in Table 2, which demonstrates the neutralization potency for each variant of the tested EGP inhibitors. As shown, the results indicate that both the unfractionated heparin and heparin enriched for high molecular weight forms are potent inhibitors. In contrast, pro-piece is clearly less potent. The glutamic and aspartic polyamino acids are remarkably potent inhibitors, and their potencies vary with their molecular weights.

TABLE 2
eMBP1 Neutralization Potencies for several EGP inhibitors.

| Inhibitor | MW (kDa) | IC50 (μg/mL) | IC50 95% CI | Hill Slope | Hill Slope 95% CI | R² | IC50 (M) |
|---|---|---|---|---|---|---|---|
| LMWH | 4.5 | 7.861 | 6.438 to 9.584 | 2.051 | 1.755 to 2.466 | 0.9882 | 1.75 × 10⁻⁶ |
| UFH | 15 | 8.226 | 7.172 to 9.473 | 2.193 | 1.962 to 2.495 | 0.9930 | 5.48 × 10⁻⁷ |
| HMWH | 23 | 6.600 | 5.408 to 7.921 | 1.864 | 1.586 to 2.298 | 0.9942 | 2.87 × 10⁻⁷ |
| WT Pro-Piece | 25.3 | 49.910 | ? | 8.157 | ? | 0.9884 | 1.97 × 10⁻⁶ |
| α poly-L-Asp | 1.15 | 14.3 | 12.66 to 16.22 | 2.808 | 2.175 to 3.904 | 0.9834 | 1.24 × 10⁻⁵ |
| | 5.75 | 4.817 | 4.258 to 5.431 | 2.929 | 2.405 to 3.746 | 0.9888 | 8.38 × 10⁻⁷ |
| | 23 | 4.301 | 3.564 to 4.905 | 3.061 | 2.357 to 6.395 | 0.9849 | 1.87 × 10⁻⁷ |
| α poly-D-Glu | 33 | 5.197 | ? to 7.172 | 2.445 | 1.748 to ? | 0.9891 | 1.57 × 10⁻⁷ |
| α poly-L-Glu | 2.6 | 6.965 | 6.080 to 7.936 | 2.581 | 1.996 to 3.705 | 0.9832 | 2.68 × 10⁻⁶ |
| | 7.5 | 6.158 | 5.081 to ? | 3.202 | 2.240 to ? | 0.9706 | 8.21 × 10⁻⁷ |
| | 9 | 5.961 | 5.187 to 6.990 | 3.499 | 2.754 to 5.019 | 0.9867 | 6.62 × 10⁻⁷ |
| | 33 | 5.118 | 4.436 to 5.888 | 2.786 | 2.159 to 3.692 | 0.9816 | 1.55 × 10⁻⁷ |
| | 120 | 4.525 | 4.072 to 5.036 | 2.621 | 2.171 to 3.286 | 0.9895 | 3.77 × 10⁻⁸ |
| γ poly-Glu | 1.2 | 19.6000 | 17.74 to 21.63 | 2.177 | 1.838 to 2.592 | 0.9896 | 1.63 × 10⁻⁵ |
| | <10 | 4.62 | 3.840 to 5.562 | 2.534 | 1.846 to 4.006 | 0.9692 | >4.62 × 10⁻⁷ |
| | >700 | 3.317 | ? | 11.5 | 3.234 to ? | 0.9861 | <4.74 × 10⁻⁹ |

LMWH = low molecular weight heparin; UFH = unfractionated heparin; HMWH = high molecular weight heparin; WT Pro-Piece = whole transcriptome pro-piece; α poly-L-Asp = α poly-L-aspartic acid; α poly-D-Glu = α poly-D-glutamic acid; α poly-L-Glu = α poly-L-glutamic acid; γ poly-Glu = γ polyglutamic acid. For each inhibitor, this table shows molecular weight in kDa, IC50 concentration in μg/mL, IC50 95% confidence interval (CI) range, hill slope, hill slope 95% CI range, R² value, and IC50 molar concentration.

With respect to aspartate, FIG. 1 depicts the potencies of several forms of aspartate for inhibiting eMBP1 in accordance with an embodiment. The results indicate that both the unfractionated heparin and heparin enriched for high molecular weight forms are potent inhibitors; in contrast, PP is clearly less potent. The glutamic and aspartic polyamino acids are remarkably potent inhibitors. As shown, monomer aspartic acid was devoid of eMBP1 inhibitory activity, and the inhibitory activity of polyaspartic acid improved as molecular weight increased. With respect to glutamate, both D- and L-amino acid polymers of glutamic acid were shown to be effective, as were the α and γ forms of glutamic acid polymers (see Table 2). Comparison of the potencies of glutamic and aspartic acids IC50 μg/ml values suggest that polyaspartic acids may have enhanced inhibitory potency.

Although some data suggests an apparent plateau in the inhibitory potencies of polyaspartic acids as molecular weight is increased (e.g., the 5.75 kDa and the 23 kDa polymers showed relatively similar activity), it is noteworthy the most active variants of polyglutamic acid were the 120 kDa and >700 kDa polymers, suggesting that particularly high molecular weight may be advantageous.

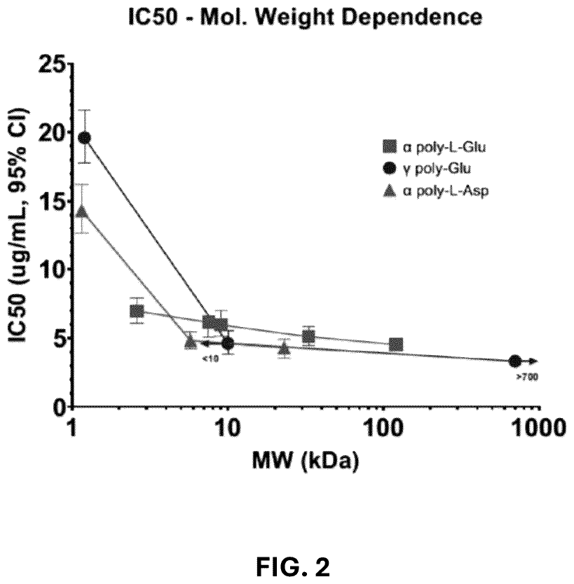

FIG. 2 depicts the half-maximal inhibitory concentration (IC50) for inhibiting eMBP1 as a function of molecular weight for each of several acidic polymers, i.e., α poly-L-glutamic acid, γ polyglutamic acid, and α poly-L-aspartic acid. IC50 is a measure of the potency of a substance in inhibiting a specific biological or biochemical function (i.e., eMBP1 in the instant case). The data informs the efficacy of the acidic polymers for inhibiting eMBP1 and also demonstrates the apparent plateau or diminishing dependence of the inhibitory potency on molecular weight as molecular weight is increased.

As shown, the IC50 (in μg/mL with 95% confidence intervals) generally decreases as molecular weight (in kDa) increases. In other words, acidic polymers with high molecular weights achieved 50% inhibition of eMBP1 at relatively lower concentrations, which signals greater binding affinity and/or avidity, and thus greater potency, for inhibiting eMBP1.

Still further, while the IC50 in μg/mL is still weight-dependent, the IC50 in molar concentration may be of greater significance to diagnostic and therapeutic applications because it elucidates an ‘absolute’ potency of the molecule (i.e., the potency on a “per molecule” basis). Accordingly, the IC50 in molar concentration (M) provides a clearer contrast of the potency of the polymers at each molecular weight. As shown in the final column of Table 2, the IC50 (M) is consistently and significantly reduced as molecular weight increases (even when comparing the same acid), indicating greater binding affinity and/or avidity for inhibiting eMBP1. In many cases, the IC50 (M) was reduced by one or more orders of magnitude, demonstrating that a significantly lower number of the molecule of acidic polymer are required to reach the IC50.

### Example 2: Investigation of Ability of Acidic Polymers for Inhibiting eMBP1-Stimulated Histamine Release from Mast Cells

Methods: To determine if the compounds shown in Table 2 had the ability to inhibit eMBP1 in a different assay, the compounds were tested for their ability to inhibit eMBP1-stimulated histamine release from mast cells. Peritoneal cells from male rats were harvested, enriched by centrifugation through a cushion of 38% bovine serum albumin (with purities up to about 90%), and stimulated with eMBP1. As expected from previous work, eMBP1 at a concentration of 10⁻⁶ M stimulated histamine release. Specifically, approximately 63% of total histamine was released (as determined by boiling the rat mast cells), and 84% was released in comparison to the amount released by compound 48/80 at 0.1 μg/ml.

In order to test inhibition of eMBP1-stimulated histamine release, each inhibitor was provided as a concentration of 50 μg/ml. Polyglutamic acid (Sigma P4761) in the molecular weight range of 15-50 kDa was utilized for testing.

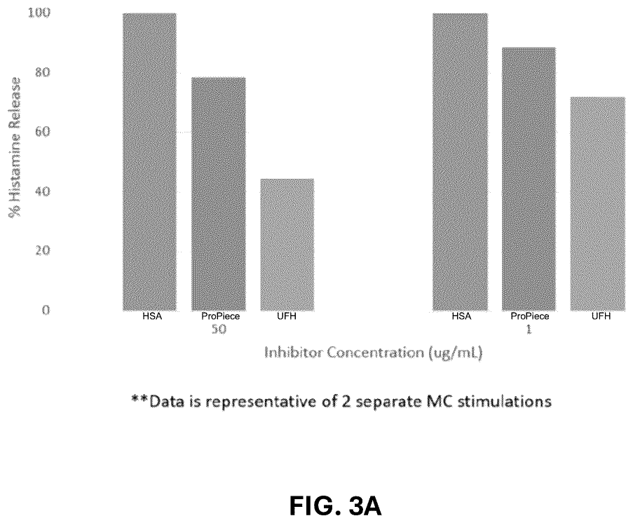

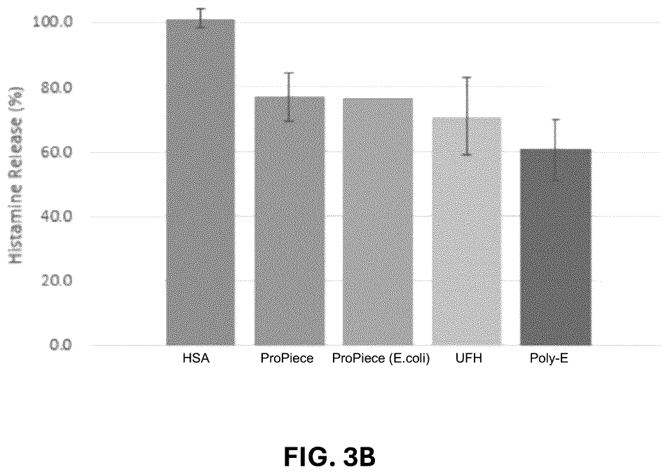

Results: FIGS. 3A-3B depict the inhibition of eMBP1-stimulated histamine release by various compounds in accordance with an embodiment. FIG. 3A shows inhibition of eMBP1-stimulated histamine release to different degrees by glycosylated pro-piece from HEK293 cells and unfractionated heparin as compared to a control protein, human serum albumin (HAS). FIG. 3B shows inhibition of eMBP1-stimulated histamine release to different degrees by glycosylated pro-piece from HEK293 cells, unglycosylated pro-piece from E. Coli, unfractionated heparin, and polyglutamic acid.

As shown, polyglutamic acid inhibited eMBP1-stimulated histamine release roughly comparably and more potently than unfractionated heparin and both glycosylated and unglycosylated pro-piece. Overall, these results demonstrate the inhibitory effects of acidic polymers on the cytotoxicity of eMBP1, including specifically for K562 tumor cells.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions, or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed is:

1. A method of treating a tissue exhibiting eosinophil-related inflammation in a subject, the method comprising administering to the subject a composition comprising:
a therapeutically effective amount of a polymer of an acidic amino acid or a pharmaceutically acceptable salt thereof, wherein the polymer has an average molecular weight of at least about 5 kDa; and
a pharmaceutically acceptable excipient,
wherein the polymer binds to one or more eosinophil granule proteins in the tissue to treat the eosinophil-related inflammation.

2. The method of claim 1, wherein the acidic amino acid is selected from the group consisting of aspartate, glutamate, and combinations thereof.

3. The method of claim 2, wherein the polymer of glutamate is selected from the group consisting of α poly-L-glutamic acid, α poly-D-glutamic acid, γ polyglutamic acid, and combinations thereof.

4. The method of claim 3, wherein the average molecular weight of the polymer of α poly-L-glutamic acid is selected from the group consisting of: about 5 kDa to about 7.5 kDa, about 7.5 kDa to about 9 kDa, about 9 kDa to about 33 kDa, about 33 kDa to about 120 kDa, and greater than about 120 kDa.

5. The method of claim 3, wherein the average molecular weight of the polymer of α poly-D-glutamic acid is selected from the group consisting of: about 5 kDa to about 33 kDa, and greater than about 33 kDa.

6. The method of claim 3, wherein the average molecular weight of the polymer of γ polyglutamic acid is selected from the group consisting of: about 5 kDa to about 10 kDa, about 10 kDa to about 50 kDa, about 50 kDa to about 100 kDa, about 100 kDa to about 500 kDa, about 500 kDa to about 700 kDa, and greater than about 700 kDa.

7. The method of claim 2, wherein the polymer of aspartate is α poly-L-aspartic acid.

8. The method of claim 7, wherein the average molecular weight of the polymer of α poly-L-aspartic acid is selected from the group consisting of: about 5 kDa to about 5.75 kDa, about 5.75 kDa to about 23 kDa, and greater than about 23 kDa.

9. The method of claim 1, wherein the average molecular weight of the polymer is at least about 10 kDa.

10. The method of claim 9, wherein the average molecular weight of the polymer is at least about 20 kDa.

11. The method of claim 10, wherein the average molecular weight of the polymer is at least about 50 kDa.

12. The method of claim 11, wherein the average molecular weight of the polymer is at least about 100 kDa.

13. The method of claim 12, wherein the average molecular weight of the polymer is at least about 500 kDa.

14. The method of claim 13, wherein the average molecular weight of the polymer is at least about 700 kDa.

15. The method of claim 1, wherein the acidic amino acid is aspartate, wherein the average molecular weight of the polymer is at least about 20 kDa.

16. The method of claim 1, wherein the acidic amino acid is glutamate, wherein the average molecular weight of the polymer is at least about 100 kDa.

17. The method of claim 16, wherein the average molecular weight of the polymer is at least about 700 kDa.

18. The method of claim 1, wherein the one or more eosinophil granule proteins comprise one or more of major basic protein 1 (eMBP1), major basic protein 2 (eMBP2), eosinophil derived neurotoxin (EDN), eosinophil cationic protein (ECP), and eosinophil peroxidase (EPO).

19. The method of claim 1, further comprising a therapeutic agent conjugated to the polymer.

20. The method of claim 19, wherein the therapeutic agent is a glucocorticoid.

21. The method of claim 1, wherein at least 50% of chains of the polymer in the composition have a molecular weight of at least 5 kDa.

22. The method of claim 21, wherein at least 60% of chains of the polymer in the composition have a molecular weight of at least 5 kDa.

23. The method of claim 22, wherein at least 70% of chains of the polymer in the composition have a molecular weight of at least 5 kDa.

24. The method of claim 1, wherein the composition is administered systemically.

25. A method of reducing eosinophil-related inflammation in a tissue in a subject, the method comprising administering to the subject a composition comprising:
a therapeutically effective amount of a polymer of an acidic amino acid or a pharmaceutically acceptable salt thereof, wherein the polymer has an average molecular weight of at least about 5 kDa; and
a pharmaceutically acceptable excipient,
wherein the polymer binds to one or more eosinophil granule proteins in the tissue to reduce the eosinophil-related inflammation.

26. The method of claim 25, wherein the acidic amino acid is selected from the group consisting of aspartate, glutamate, and combinations thereof.

27. The method of claim 26, wherein the polymer of glutamate is selected from the group consisting of α poly-L-glutamic acid, α poly-D-glutamic acid, γ polyglutamic acid, and combinations thereof.

28. The method of claim 27, wherein the average molecular weight of the polymer of a poly-L-glutamic acid is selected from the group consisting of: about 5 kDa to about 7.5 kDa, about 7.5 kDa to about 9 kDa, about 9 kDa to about 33 kDa, about 33 kDa to about 120 kDa, and greater than about 120 kDa.

29. The method of claim 27, wherein the average molecular weight of the polymer of a poly-D-glutamic acid is selected from the group consisting of: about 5 kDa to about 33 kDa, and greater than about 33 kDa.

30. The method of claim 27, wherein the average molecular weight of the polymer of γ polyglutamic acid is selected from the group consisting of: about 5 kDa to about 10 kDa, about 10 kDa to about 50 kDa, about 50 kDa to about 100 kDa, about 100 kDa to about 500 kDa, about 500 kDa to about 700 kDa, and greater than about 700 kDa.

31. The method of claim 26, wherein the polymer of aspartate is α poly-L-aspartic acid.

32. The method of claim 31, wherein the average molecular weight of the polymer of a poly-L-aspartic acid is selected from the group consisting of: about 5 kDa to about 5.75 kDa, about 5.75 kDa to about 23 kDa, and greater than about 23 kDa.

33. The method of claim 25, wherein the average molecular weight of the polymer is at least about 10 kDa.

34. The method of claim 33, wherein the average molecular weight of the polymer is at least about 20 kDa.

35. The method of claim 34, wherein the average molecular weight of the polymer is at least about 50 kDa.

36. The method of claim 35, wherein the average molecular weight of the polymer is at least about 100 kDa.

37. The method of claim 36, wherein the average molecular weight of the polymer is at least about 500 kDa.

38. The method of claim 37, wherein the average molecular weight of the polymer is at least about 700 kDa.

39. The method of claim 25, wherein the acidic amino acid is aspartate, wherein the average molecular weight of the polymer is at least about 20 kDa.

40. The method of claim 25, wherein the acidic amino acid is glutamate, wherein the average molecular weight of the polymer is at least about 100 kDa.

41. The method of claim 40, wherein the average molecular weight of the polymer is at least about 700 kDa.

42. The method of claim 25, wherein the one or more eosinophil granule proteins comprise one or more of major basic protein 1 (eMBP1), major basic protein 2 (eMBP2), eosinophil derived neurotoxin (EDN), eosinophil cationic protein (ECP), and eosinophil peroxidase (EPO).

43. The method of claim 25, further comprising a therapeutic agent conjugated to the polymer.

44. The method of claim 43, wherein the therapeutic agent is a glucocorticoid.

45. The method of claim 25, wherein at least 50% of chains of the polymer in the composition have a molecular weight of at least 5 kDa.

46. The method of claim 45, wherein at least 60% of chains of the polymer in the composition have a molecular weight of at least 5 kDa.

47. The method of claim 46, wherein at least 70% of chains of the polymer in the composition have a molecular weight of at least 5 kDa.

48. The method of claim 25, wherein the composition is administered systemically.

49. A composition for use in treating a tissue exhibiting eosinophil-related inflammation in a subject, the composition comprising:
an effective amount of a polymer of an acidic amino acid or a pharmaceutically acceptable salt thereof, the polymer having an average molecular weight of at least about 5 kDa, wherein the polymer is configured to bind to one or more eosinophil granule proteins in the tissue to treat the eosinophil-related inflammation; and
a pharmaceutically acceptable excipient.

50. The composition of claim 49, wherein the acidic amino acid is selected from the group consisting of aspartate, glutamate, and combinations thereof.

51. The composition of claim 50, wherein the polymer of glutamate is selected from the group consisting of α poly-L-glutamic acid, α poly-D-glutamic acid, γ polyglutamic acid, and combinations thereof.

52. The composition of claim 51, wherein:
the average molecular weight of the polymer of α poly-L-glutamic acid is selected from the group consisting of: about 5 kDa to about 7.5 kDa, about 7.5 kDa to about 9 kDa, about 9 kDa to about 33 kDa, about 33 kDa to about 120 kDa, and greater than about 120 kDa; or
the average molecular weight of the polymer of α poly-D-glutamic acid is selected from the group consisting of: about 5 kDa to about 33 kDa, and greater than about 33 kDa; or
the average molecular weight of the polymer of γ polyglutamic acid is selected from the group consisting of: about 5 kDa to about 10 kDa, about 10 kDa to about 50 kDa, about 50 kDa to about 100 kDa, about 100 kDa to about 500 kDa, about 500 kDa to about 700 kDa, and greater than about 700 kDa.

53. The composition of claim 50, wherein the polymer of aspartate is α poly-L-aspartic acid, wherein the average molecular weight of the polymer of α poly-L-aspartic acid is selected from the group consisting of: about 5 kDa to about 5.75 kDa, about 5.75 kDa to about 23 kDa, and greater than about 23 kDa.

54. The composition of claim 49, wherein the average molecular weight of the polymer is selected from the group consisting of: at least about 10 kDa, at least about 20 kDa, at least about 50 kDa, at least about 100 kDa, at least about 500 kDa, and at least about 700 kDa.

55. The composition of claim 49, further comprising a therapeutic agent conjugated to the polymer, wherein the therapeutic agent is a glucocorticoid.

56. The composition of claim 49, wherein at least 50% of chains of the polymer in the composition have a molecular weight of at least 5 kDa.

57. The composition of claim 49, wherein the composition is administered systemically.

58. The composition of claim 49, further comprising a radiolabeled contrast agent conjugated to the polymer.

59. The composition of claim 50, wherein the radiolabeled contrast agent is ^{99m}Tc.

60. A method of producing a medical image of an organ in a subject, the method comprising:
administering, to the subject, a composition comprising an effective dose of a radiolabeled polymer of an acidic amino acid or a pharmaceutically acceptable salt thereof and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and
detecting the radiolabeled polymer/eosinophil granule protein complex in a mucosal tissue of the organ, thereby producing a medical image of the organ.

61. The method of claim 60, wherein the radiolabeled polymer/eosinophil granule protein complex is detected using single-photon emission computed tomography (SPECT), positron emission tomography (PET), conventional or computed tomography (CT), magnetic resonance imaging (MRI), or a combination thereof.

62. The method claim 60, wherein the acidic amino acid is selected from the group consisting of aspartate, glutamate, and combinations thereof.

63. The method of claim 62, wherein the polymer of glutamate is selected from the group consisting of α poly-L-glutamic acid, α poly-D-glutamic acid, γ polyglutamic acid, and combinations thereof.

64. The method of claim 62, wherein the polymer of aspartate is α poly-L-aspartic acid.

65. The method of claim 60, wherein the radiolabeled polymer comprises a radiolabeled contrast agent conjugated to a polymer.

66. The method of claim 65, wherein the radiolabeled contrast agent is ^{99m}Tc.

67. The method of claim 60, wherein the composition is administered systemically.

68. A method of diagnosing eosinophil-related inflammation in an organ of a subject, the method comprising:
administering, to the subject, a composition comprising an effective dose of a radiolabeled polymer of an acidic amino acid or a pharmaceutically acceptable salt thereof and a pharmaceutically acceptable excipient, the radiolabeled polymer having an average molecular weight of at least about 5 kDa, wherein the radiolabeled polymer is configured to bind to one or more eosinophil granule proteins to form a radiolabeled polymer/eosinophil granule protein complex, and
detecting the radiolabeled polymer/eosinophil granule protein complex in a mucosal tissue of the organ, thereby diagnosing the eosinophil-related inflammation in the organ.

69. The method of claim 68, wherein the radiolabeled polymer/eosinophil granule protein complex is detected using single-photon emission computed tomography (SPECT), positron emission tomography (PET), conventional or computed tomography (CT), magnetic resonance imaging (MRI), or a combination thereof.

70. The method claim 68, wherein the acidic amino acid is selected from the group consisting of aspartate, glutamate, and combinations thereof.

71. The method of claim 70, wherein the polymer of glutamate is selected from the group consisting of α poly-L-glutamic acid, α poly-D-glutamic acid, γ polyglutamic acid, and combinations thereof.

72. The method of claim 70, wherein the polymer of aspartate is α poly-L-aspartic acid.

73. The method of claim 68, wherein the radiolabeled polymer comprises a radiolabeled contrast agent conjugated to a polymer.

74. The method of claim 73, wherein the radiolabeled contrast agent is ^{99m}Tc.

75. The method of claim 68, wherein the composition is administered systemically.
